(19) United States
(12) Patent Application Publication
(10) Pub. No.: US 2024/0108221 A1
(43) Pub. Date: Apr. 4, 2024

(54) METHOD AND SYSTEM FOR PROVIDING DATA MANAGEMENT IN INTEGRATED ANALYTE MONITORING AND INFUSION SYSTEM

(71) Applicant: Abbott Diabetes Care, Inc., Alameda, CA (US)

(72) Inventor: R. Curtis JENNEWINE, San Francisco, CA (US)

(73) Assignee: Abbott Diabetes Care, Inc., Alameda, CA (US)

(21) Appl. No.: 18/478,676

(22) Filed: Sep. 29, 2023

Related U.S. Application Data
(63) Continuation of application No. 17/947,628, filed on Sep. 19, 2022, now Pat. No. 11,806,110.
Application No. 17/947,628 is a continuation of application No. 15/640,632, filed on Jul. 3, 2017, now Pat. No. 11,445,910.
Application No. 15/640,632 is a continuation of application No. 14/563,658, filed on Dec. 8, 2014, now Pat. No. 9,697,332.
Application No. 14/563,658 is a continuation of application No. 11/462,982, filed on Aug. 7, 2006, now Pat. No. 8,932,216.

(51) Int. Cl. A61B 5/00; A61B 5/145; G16H 20/17; G16H 40/67

(52) U.S. Cl. CPC A61B 5/0031; A61B 5/14532; A61B 5/14546; A61B 5/4839; A61B 5/7275; A61B 5/743; G16H 20/17; G16H 40/67

(57) ABSTRACT

Methods and systems for providing therapy related data management are provided. The subject systems include one or more device components, and at least one memory storage unit and at least one data storage unit associated with such one or more device components. The device components may include one or more of an analyte monitoring system, a fluid delivery device and a remote terminal. The subject methods include use of the subject systems to optimize treatment of a patient.

### CROSS-REFERENCE

The present application is a continuation of U.S. patent application Ser. No. 17/947,628, filed Sep. 19, 2022, which is a continuation of U.S. patent application Ser. No. 15/640,632 filed Jul. 3, 2017, now U.S. Pat. No. 11,445,910, which is a continuation of U.S. patent application Ser. No. 14/563,658 filed Dec. 8, 2014, now U.S. Pat. No. 9,697,332, which is a continuation of U.S. patent application Ser. No. 11/462,982 filed Aug. 7, 2006, now U.S. Pat. No. 8,932,216, all entitled “Method and System for Providing Data Management in Integrated Analyte Monitoring and Infusion System,” the disclosure of both are incorporated herein by reference for all purposes.

### BACKGROUND

With increasing use of pump therapy for Type 1 diabetic patients, young and old alike, the importance of controlling the infusion device such as external infusion pumps is evident. Indeed, presently available external infusion devices typically include an input mechanism such as buttons through which the patient may program and control the infusion device. Such infusion devices also typically include a user interface such as a display which is configured to display information relevant to the patient's infusion progress, status of the various components of the infusion device, as well as other programmable information such as patient specific basal profiles.

The external infusion devices are typically connected to an infusion set which includes a cannula that is placed transcutaneously through the skin of the patient to infuse a select dosage of insulin based on the infusion device's programmed basal rates or any other infusion rates as prescribed by the patient's doctor. Generally, the patient is able to control the pump to administer additional doses of insulin during the course of wearing and operating the infusion device such as for, administering a carbohydrate bolus prior to a meal. Certain infusion devices include food database that has associated therewith, an amount of carbohydrate, so that the patient may better estimate the level of insulin dosage needed for, for example, calculating a bolus amount.

However, in general, most estimation or calculation of a bolus amount for administration, or a determination of a suitable basal profile, for that matter, are educated estimates based on the patient's physiology as determined by the patient's doctor, or an estimate performed by the patient. Moreover, the infusion devices do not generally include enhancement features that would better assist the diabetic patients to control and/or manage the glucose levels.

In view of the foregoing, it would be desirable to have an approach to provide methods and system for data processing associated with a patient's monitored analyte levels for providing semi automatic or automatic recommendation based on the processed data such as, for example, therapy profile, glucose level pattern recognition, and the like.

### SUMMARY OF THE INVENTION

In accordance with one embodiment of the present invention, there is provided method and system for detecting a predetermined rate of increase in an analyte level of a patient over a predefined time period, and associating a temporal identifier associated with the detected predetermined rate of increase in the analyte level of the patient.

These and other objects, features and advantages of the present invention will become more fully apparent from the following detailed description of the embodiments, the appended claims and the accompanying drawings.

### BRIEF DESCRIPTION OF THE DRAWINGS

FIG. 1 is a block diagram illustrating a therapy management system for practicing one embodiment of the present invention;

FIG. 2 is a block diagram of an fluid delivery device of FIG. 1 in one embodiment of the present invention;

FIG. 3 is a block diagram illustrating the remote terminal of FIG. 1 for performing data processing in accordance with one embodiment of the present invention;

FIG. 4 is a flow % chart illustrating analyte level associated data processing related to meals in accordance with one embodiment of the present invention;

FIG. 5 is a flowchart illustrating therapy management related data processing in accordance with one embodiment of the present invention; and

FIG. 6 is a flowchart illustrating time of day related data processing associated with meal events in accordance with one embodiment of the present invention.

### DETAILED DESCRIPTION

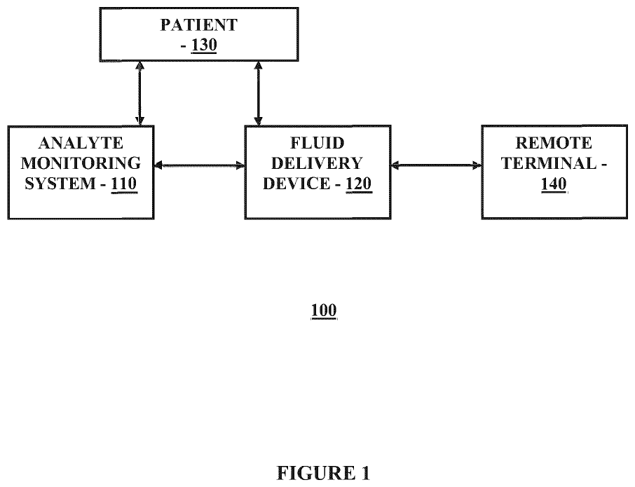

FIG. 1 is a block diagram illustrating an insulin therapy management system for practicing one embodiment of the present invention. Referring to FIG. 1, the therapy management system 100 includes an analyte monitoring system 110 operatively coupled to an fluid delivery device 120, which maw be in turn operatively coupled to a remote terminal 140. As shown the Figure, the analyte monitoring s stem 110 is, in one embodiment, coupled to the patient 130 so as to monitor or measure the analyte levels of the patient. Moreover, the fluid delivery device 120 is coupled to the patient using, for example, and infusion set and tubing connected to a cannula (not shown) that is placed transcutaneously through the skin of the patient so as to infuse medication such as, for example, insulin, to the patient.

Referring to FIG. 1, in one embodiment the analyte monitoring system 110 in one embodiment may include one or more analyte sensors subcutaneously positioned such that at least a portion of the analyte sensors are maintained in fluid contact with the patient's analytes. The analyte sensors may include, but not limited to short term subcutaneous analyte sensors or transdermal analyte sensors, for example, which are configured to detect analyte levels of a patient over a predetermined time period, and after which, a replacement of the sensors is necessary.

The one or more analyte sensors of the analyte monitoring system 110 is coupled to a respective one or more of a data transmitter unit which is configured to receive one or more signals from the respective analyte sensors corresponding to the detected analyte levels of the patient, and to transmit the information corresponding to the detected analyte levels to a receiver device, and/or fluid delivery device 120. That is, over a communication link, the transmitter units may be configured to transmit data associated with the detected analyte levels periodically, and/or intermittently and repeatedly to one or more other devices such as the fluid deliver device and/or the remote terminal 140 for further data processing and analysis.

The transmitter units of the analyte monitoring sc stem 110 mac in one embodiment configured to transmit the analyte related data substantially in real time to the fluid delivery device 120 and/or the remote terminal 140 after receiving it from the corresponding analyte sensors such that the analyte level such as glucose level of the patient 130 may be monitored in real time. In one aspect, the analyte levels of the patient may be obtained using one or more of a discrete blood glucose testing devices such as blood glucose meters, or a continuous analyte monitoring systems such as continuous glucose monitoring systems.

Additional analytes that may be monitored, determined or detected the analyte monitoring system 110 include, for example, acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be determined.

Moreover, within the scope of the present invention, the transmitter units of the analyte monitoring system 110 may be configured to directly communicate with one or more of the remote terminal 140 or the fluid delivery device 120. Furthermore, within the scope of the present invention, additional devices may be provided for communication in the therapy management system 100 including additional receiver/data processing unit, remote terminals (such as a physician's terminal and/or a bedside terminal in a hospital environment, for example. In addition, within the scope of the present invention, one or more of the analyte monitoring system 110, the fluid delivery device 120 and the remote terminal 140 may be configured to communicate over a wireless data communication link such as, but not limited to RF communication link. Bluetooth communication link, infrared communication link, or any other type of suitable wireless communication connection between two or more electronic devices, which may further be uni-directional or bi-directional communication between the two or more devices. Alternatively, the data communication link may include wired cable connection such as, for example, but not limited to RS232 connection, USB connection, or serial cable connection.

The fluid delivery device 120 may include in one embodiment, but not limited to, an external infusion device such as an external insulin infusion pump, an implantable pump, a pen-type insulin injector device, a patch pump, an inhalable infusion device for nasal insulin delivery, or any other type of suitable delivery system. In addition, the remote terminal 140 in one embodiment may include for example, a desktop computer terminal, a data communication enabled kiosk, a laptop computer, a handheld computing device such as a personal digital assistant (PDAs), or a data communication enabled mobile telephone.

Referring back to FIG. 1, in one embodiment, the analyte monitoring system 100 includes a strip port configured to receive a test strip for capillary blood glucose testing. In one aspect, the glucose level measured using the test strip may in addition, be configured to provide periodic calibration of the analyte sensors of the analyte monitoring system 100 to assure and improve the accuracy of the analyte levels detected by the analyte sensors.

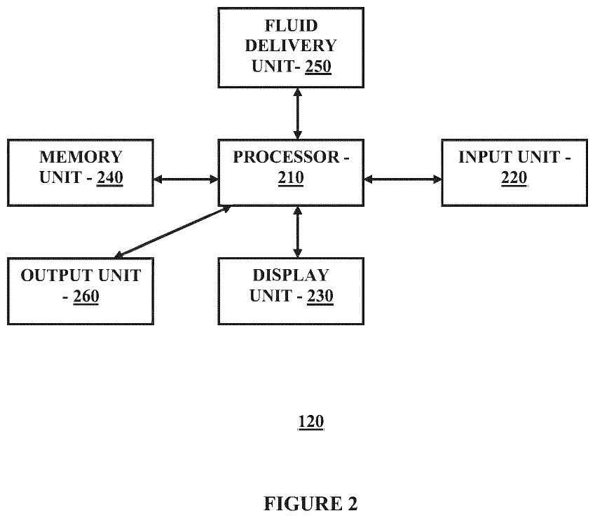

FIG. 2 is a block diagram of an fluid delivery device of FIG. 1 in one embodiment of the present invention. Referring to FIG. 2, the fluid delivery device 120 in one embodiment includes a processor 210 operatively coupled to a memory unit 240, an input unit 220, a display unit 230, an output unit 260, and a fluid delivery unit 250. In one embodiment, the processor 210 includes a microprocessor that is configured to and capable of controlling the functions of the fluid delivery device 120 by controlling and/or accessing each of the various components of the fluid delivery device 120. In one embodiment, multiple processors may be provided as safety measure and to provide redundancy in case of a single processor failure. Moreover, processing capabilities may be shared between multiple processor units within the fluid delivery device 120 such that pump functions and/or control maybe performed faster and more accurately.

Referring back to FIG. 2, the input unit 220 operatively coupled to the processor 210 may include a jog dial, an key pad buttons, a touch pad screen, or any other suitable input mechanism for providing input commands to the fluid delivery device 120. More specifically, in case of a jog dial input device, or a touch pad screen, for example, the patient or user of the fluid delivery device 120 will manipulate the respective jog dial or touch pad in conjunction with the display unit 230 which performs as both a data input and output units. The display unit 230 may include a touch sensitive screen, an LCD screen, or any other types of suitable display unit for the fluid delivery device 120 that is configured to display alphanumeric data as well as pictorial information such as icons associated with one or more predefined states of the fluid delivery device 120, or graphical representation of data such as trend charts and graphs associated with the insulin infusion rates, trend data of monitored glucose levels over a period of time, or textual notification to the patients.

Referring to FIG. 2, the output unit 260 operatively coupled to the processor 210 may include audible alarm including one or more tones and//or preprogrammed or programmable tunes or audio clips, or vibratory alert features having one or more pre-programmed or programmable vibratory alert levels. In one embodiment, the vibratory alert may also assist in priming the infusion tubing to minimize the potential for air or other undesirable material in the infusion tubing. Also shown in FIG. 2 is the fluid delivery unit 250 which is operatively coupled to the processor 210 and configured to deliver the insulin doses or amounts to the patient from the insulin reservoir or any other types of suitable containment for insulin to be delivered (not shown) in the fluid delivery device 120 via an infusion set coupled to a subcutaneously positioned cannula under the skin of the patient.

Referring yet again to FIG. 2, the memory unit 240 may include one or more of a random access memory (RAM), read only memory (ROM), or any other types of data storage units that is configured to store data as well as program instructions for access by the processor 210 and execution to control the fluid delivery device 120 and/or to perform data processing based on data received from the analyte monitoring system 110, the remote terminal 140, the patient 130 or any other data input source.

Within the scope of the present invention, the fluid delivery device 120 may be configured to ascertain these consecutive glucose readings from the data stream received from the analyte monitoring system 110. Moreover, the predefined time period may additionally include any other suitable time period where the monitored analyte levels may provide information associated with the patient's physiological condition as pertains to the insulin therapy and diabetes management. For example, the predefined time period may include a 4-7 day period (or longer or shorter as may be appropriate), where the fluid delivery device 120 may be configured to receive the glucose readings at a specific time of the day (for example, at 7 am in the morning). In this case, the consecutive glucose readings may include each measured glucose level at 7 am in the morning for the 4-7 day period.

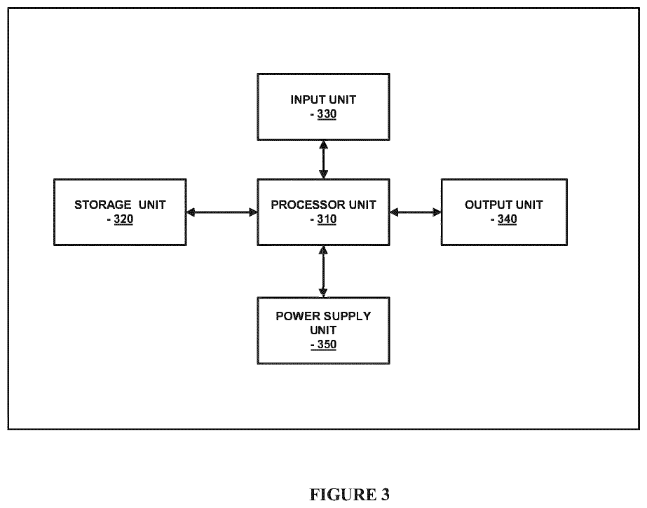

FIG. 3 is a block diagram illustrating the remote terminal of FIG. 1 for performing data processing in accordance with one embodiment of the present invention. Referring to FIG. 3, the remote terminal 140 in one embodiment includes a processor unit 310 operatively coupled to a storage unit 320. The storage unit 320 in one embodiment includes one or more of a volatile memory and non-volatile memory for storing, for example, program instructions associated with the operation of the remote terminal 140 for execution by the processor unit 310, and further, for storing analyte related data as well as infusion related data.

Referring back to FIG. 3, the processor unit 310 is further operatively coupled to an input unit 330, and an output unit 340, as well as a power supply unit 350. In one embodiment, the power supply unit 350 is configured to provide power to each of the components of the remote terminal 140 described above, and further, to effectively manage power based on instructions received from the processor unit 310. The input unit 330 may be configured to receive one or more instructions, commands or data from a patient via, for example, a keyboard device, a mouse device, or any other similar type of input devices, and for providing the received one or more instructions, commands or information, to the processor unit 310 for further processing and control.

Additionally, the output unit 340 in one embodiment is configured to output one or more data or information under the control of the processor unit 310. For example, in one embodiment, the output unit 340 may include a visual output unit such as a display unit, an audio output unit, such as a speaker unit, or a tactile output unit, such as a vibratory unit. In one embodiment, one two or more of such output components may be combined to either sequentially or simultaneously output the corresponding output signals under the command and control of the processor unit 310.

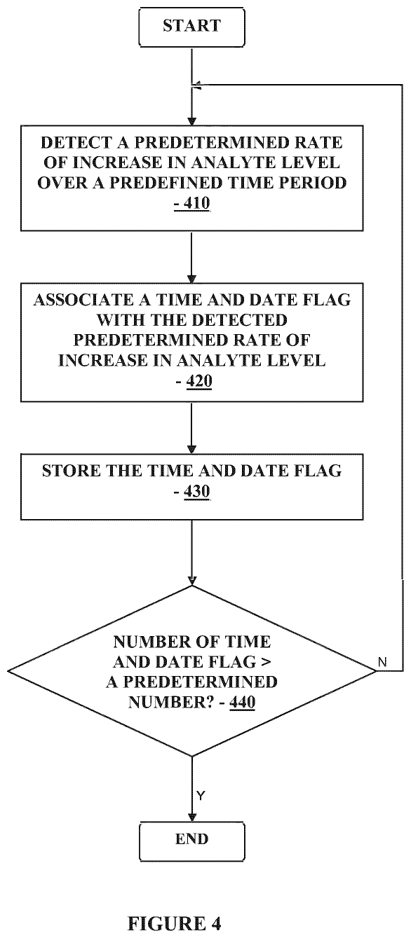

FIG. 4 is a flowchart illustrating analyte level associated data processing related to meals in accordance with one embodiment of the present invention. Referring to FIG. 4, a predetermined rate of increase in analyte level over a predefined time period is detected. For example, the rate of increase in the analyte level may be predefined or preprogrammed in the processor unit 310 (FIG. 3) to correspond or correlate to a meal event. That is, in the event that a rapid peak or sudden spike is detected in the monitored analyte level, the processor unit 310 is configured associate such event with a predefined meal event.

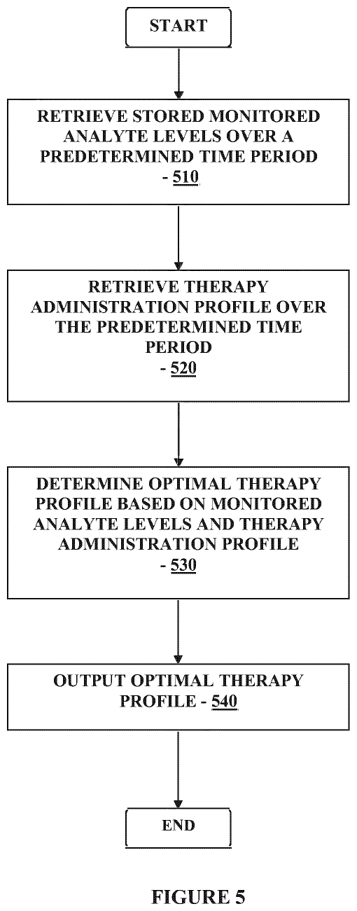

Referring back to FIG. 5, thereafter, the detected predetermined rate of increase in analyte level is associated with a time and date flag. Then, the associated time and date flag is stored in a memory, for example in the storage unit 320 (FIG. 3). It is then determined whether the number of the time and data flags is greater than a predetermined number. That is, in one embodiment, it is determined whether the number of time and date flags associated with each associated predetermined rate of increase in monitored analyte levels exceeds a predetermined number. In one embodiment, the number of time and date flags is associated with the number of meals that the patient ingests during a predetermined time period. That is, in one embodiment, each rapid increase in the analyte level is associated with a meal event of the patient.

In this manner, in one embodiment, each meal event of the patient may be associated with a time and date flag and stored in a database with an associated glucose level. Accordingly, based on the monitored analyte levels, in one embodiment, each meal event may be lined up for further processing without the need for the patient to individually lineup each meal event. That is, in one embodiment of the present invention, meal related pattern recognition may be performed and executed automatically without the manual data association by the patient for each meal time and date information and the associated carbohydrate information.

FIG. 5 is a flowchart illustrating therapy management related data processing in accordance with one embodiment of the present invention. Referring to FIG. 5, the stored monitored analyte levels over a predetermined time period is retrieved. Thereafter, the therapy administration profile over the predetermined time period is retrieved. For example, in one embodiment, the retrieved therapy administration profile may include one or more, but not limited to, stored basal profiles, carbohydrate and/or correction bolus amount administered, temporary basal profiles administered, and the like.

Referring again to FIG. 5, an optimal therapy profile is determined based on the retrieved monitored analyte levels and the therapy administration profile. Thereafter, the determined optimal therapy profile is output, for example, on the display unit 340 (FIG. 3) for review and/or further analysis by the patient, or the patient's healthcare provider. In this manner, in one embodiment of the present invention, it is possible to provide an automated pattern recognition for therapy administration profiles such as basal profiles and/or bolus amounts, to assist the healthcare providers in analyzing the monitored analyte levels in conjunction with the executed therapy administration profiles.

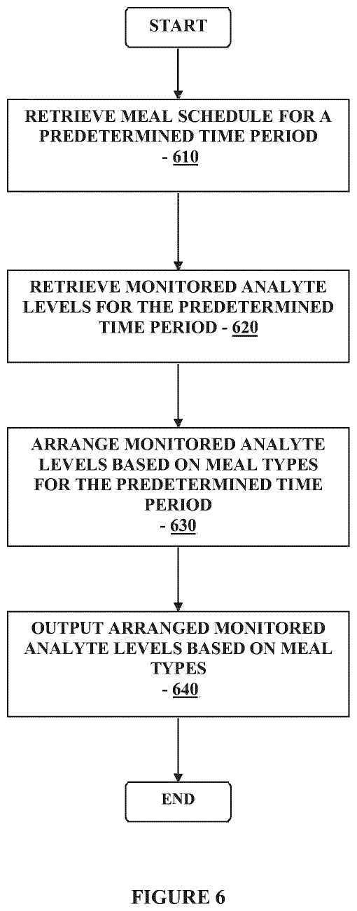

FIG. 6 is a flowchart illustrating time of day related data processing associated with meal events in accordance with one embodiment of the present invention. Referring to FIG. 6, the meal schedule of the patient for a predetermined time period is retrieved. Thereafter, the monitored analyte levels for the predetermined time period is retrieved, for example, from the storage unit 320 of the remote terminal 140. Thereafter, the monitored analyte levels are arranged or realigned based on the meal types for the predetermined time period, and the arranged or realigned monitored analyte levels are output on the display unit 340, for example.

That is, in one embodiment, based on the time of date information of a patient, and the associated monitored analyte levels of the patient, time based events may be associated with the corresponding monitored analyte levels. For example, for shift workers with irregular work schedule such as alternating day and night shifts, it would be possible to lineup meal events for such workers based on the time and date information and the associated monitored analyte levels so as to effectively and meaningfully perform data processing based on the intake of food and the monitored analyte levels.

A method in accordance with one embodiment of the present invention includes detecting a predetermined rate of increase in an analyte level of a patient over a predefined time period, and associating a temporal identifier associated with the detected predetermined rate of increase in the analyte level of the patient.

The predetermined rate of increase over a predefined time period may include a change in the analyte level exceeding a predetermined percentage over the predefined time period, where the predefined time period may include one of less than 5 minutes, less than 30 minutes, or less than one hour.

In another aspect, the predetermined percentage may include one of less than 15 percent, less than 50 percent, or less than 80 percent.

The method may also include storing the temporal identifier associated with the detected predetermined rate of increase in the analyte level of the patient.

The temporal identifier in one embodiment may include one or more of a time information or a date information.

In one aspect, the temporal identifier may include a meal event.

A method in accordance with another embodiment includes retrieving one or more stored analyte levels of a predetermined time period, retrieving one or more stored therapy administration profile for the predetermined time period, determining an optimal therapy profile based on the retrieved one or more stored analyte levels and the retrieved one or more stored therapy administration profile.

The method may include outputting the optical therapy profile.

In one embodiment, the retrieved one or more stored therapy administration profile may include a pre-programmed one or more basal profiles.

In a further aspect, the predetermined time period may include one of a one day period, a 3 day period, or less than 8 day period.

The optimal therapy profile may include, in one embodiment, a modified one or more stored therapy administration profiles.

A method in accordance with yet another embodiment of the present invention includes retrieving a meal schedule for a predetermined time period, retrieving monitored analyte levels for the predetermined time period, and arranging the monitored analyte levels based on one or more meal types of the meal schedule for the predetermined time period.

The method may also include outputting the arranged monitored analyte levels.

The meal schedule in one embodiment may include one or more time period associated with breakfast, lunch or dinner.

In a further aspect, arranging the monitored analyte levels may include associating a respective analyte level for each retrieved meal schedule.

The predetermined time period in yet a further aspect may include one or more of a 3 day period, a seven day period, or less than 14 day period.

A system for providing diabetes management in accordance with still another embodiment includes a storage unit configured to store one or more data associated with monitored analyte levels, a processor unit operatively coupled to the storage unit, and configured to perform one or more processes to detect a predetermined rate of increase in an analyte level of a patient over a predefined time period, and associate a temporal identifier associated with the detected predetermined rate of increase in the analyte level of the patient.

The predetermined rate of increase over a predefined time period includes a change in the analyte level exceeding a predetermined percentage over the predefined time period.

The various processes described above including the processes performed by the processor 210 in the software application execution environment in the fluid delivery device 120 as well as any other suitable or similar processing units embodied in the analyte monitoring system 120 and the remote terminal 140, including the processes and routines described in conjunction with FIGS. 4-6, may be embodied as computer programs developed using an object oriented language that allows the modeling of complex systems with modular objects to create abstractions that are representative of real world, physical objects and their interrelationships. The software required to carry out the inventive process, which may be stored in the memory unit 240 (or in a similar storage device in the analyte monitoring system 120 and in the storage unit 320 of the remote terminal 140 for execution by the processor unit 310) of the processor 210, may be developed by a person of ordinary skill in the art and may include one or more computer program products.

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present invention and that structures and methods within the scope of these claims and their equivalents be covered thereby.

1-20. (canceled)

21. A method for displaying a graphical representation of monitored analyte levels the method comprising:
retrieving a meal schedule associated with a user, wherein the meal schedule includes one or more meal types, and wherein the meal schedule is for a predetermined time period;
retrieving a monitored analyte level associated with the predetermined time period, wherein the monitored analyte level is based on continuous glucose sensor data from a continuous glucose sensor, and wherein the continuous glucose sensor is configured to be subcutaneously positioned such that at least a portion of the continuous glucose sensor data is determined based on the continuous glucose sensor being in fluid contact with analytes of the user;
arranging, in the graphical representation, the monitored analyte level based on the one or more meal types of the meal schedule; and
displaying the graphical representation on a display device.

22. The method of claim 21, wherein the continuous glucose sensor data comprises a rate of increase of the analyte level of the host.

23. The method of claim 21, wherein the one or more meal types is one of breakfast, lunch, or dinner.

24. The method of claim 21, wherein the predetermined time period is a time period associated with the one or more meal types.

25. The method of claim 21, wherein the predetermined time period comprises at least two time periods associated with the one or more meal types.

26. The method of claim 25, wherein the monitored analyte level comprises a plurality of monitored analyte levels and the one or more meal types comprises at least two meal types, and wherein arranging the monitored analyte level comprises arranging each analyte level of the monitored analyte levels based on the at least two meal types.

27. The method of claim 21, further comprising storing the one or more meal types and the monitored analyte level in a database.

28. The method of claim 27, further comprising:
identifying a meal event based on the one or more meal type and the monitored analyte level;
determining at least one time information or date information for the meal event based on the predetermined time period; and
storing the at least one time information or date information with the meal event level in the database.

29. A system for displaying a graphical representation of monitored analyte levels, the system comprising:
one or more processors;
computer memory; and
computer-readable instructions stored in the computer memory, wherein the instructions are configured to cause the one or more processors to:
retrieve a meal schedule associated with a user, wherein the meal schedule includes one or more meal types, and wherein the meal schedule is for a predetermined time period;
retrieve a monitored analyte level associated with the predetermined time period, wherein the monitored analyte level is based on continuous glucose sensor data from a continuous glucose sensor, and wherein the continuous glucose sensor is configured to be subcutaneously positioned such that at least a portion of the continuous glucose sensor data is determined based on the continuous glucose sensor being in fluid contact with analytes of the user;
arrange, in the graphical representation, the monitored analyte level based on the one or more meal types of the meal schedule; and
display the graphical representation on a display device.

30. The system of claim 29, wherein the continuous glucose sensor data comprises a rate of increase of the analyte level of the host.

31. The system of claim 29, wherein the one or more meal types is one of breakfast, lunch, or dinner.

32. The system of claim 29, wherein the predetermined time period is a time period associated with the one or more meal types.

33. The system of claim 9, wherein the predetermined time period comprises at least two time periods associated with the one or more meal types.

34. The system of claim 33, wherein the monitored analyte level comprises a plurality of monitored analyte levels and the one or more meal types comprises at least two meal types, and wherein arranging the monitored analyte level comprises arranging each analyte level of the monitored analyte levels based on the at least two meal types

35. The system of claim 29, further comprising storing the one or more meal types and the monitored analyte level in a database.

36. A non-transitory computer-readable medium having instructions stored thereon that, when executed by at least one computing device, cause the at least one computing device to perform operations for displaying a graphical representation of continuous glucose sensor data, the operations comprising:
retrieving a meal schedule associated with a user, wherein the meal schedule includes one or more meal types, and wherein the meal schedule is for a predetermined time period;
retrieving a monitored analyte level associated with the predetermined time period, wherein the monitored analyte level is based on continuous glucose sensor data from a continuous glucose sensor, and wherein the continuous glucose sensor is configured to be subcutaneously positioned such that at least a portion of the continuous glucose sensor data is determined based on the continuous glucose sensor being in fluid contact with analytes of the user,
arranging, in the graphical representation, the monitored analyte level based on the one or more meal types of the meal schedule; and
displaying the graphical representation on a display device.

37. The non-transitory computer-readable medium of claim 36, wherein the continuous glucose sensor data comprises a rate of increase of the analyte level of the host.

38. The non-transitory computer-readable medium of claim 36, wherein the one or more meal types is one of breakfast, lunch, or dinner.

39. The non-transitory computer-readable medium of claim 36, wherein the predetermined time period is a time period associated with the one or more meal types.

40. The non-transitory computer-readable medium of claim 39, wherein the monitored analyte level comprises a plurality of monitored analyte levels and the one or more meal types comprises at least two meal types, and wherein arranging the monitored analyte level comprises arranging each analyte level of the monitored analyte levels based on the at least two meal types
